(19) United States
(12) Patent Application Publication
(10) Pub. No.: US 2010/0189783 A1
(43) Pub. Date: Jul. 29, 2010

(54) RELATING TO ANTI-HIV TABLET FORMULATIONS

(76) Inventors: Guido Franciscus Smans, Lille (BE); Eugeen Maria Jozef Jans, Meerhout (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/670,030

(22) PCT Filed: Jul. 25, 2008
(86) PCT No.: PCT/EP08/59802

§ 371 Date: Jan. 21, 2010

(30) Foreign Application Priority Data
Jul. 25, 2007 (EP) 07113104.9

(51) Int. Cl. A61K 9/20; A61K 31/34; A61P 31/18

(52) U.S. Cl. 424/465; 514/470

(57) ABSTRACT

An anti-HIV tablet formulation comprising a core containing 0.1 to 1.5% by weight (w/w) of colloidal silicon dioxide and 0.4 to 0.9% by weight (w/w) of a lubricant, the balance of the core comprising darunavir, a disintegrant and a filler comprising a spray-dried mixture of microcrystalline cellulose and colloidal silicon dioxide, the core being optionally coated with a film coating.

The present invention relates to improved tablet formulations containing the anti-HIV agent darunavir which provide new and valuable processing properties.

### BACKGROUND

The virus causing the acquired immunodeficiency syndrome (AIDS) is known by different names, including T-lymphocyte virus III (HTLV-III) or lymphadenopathy-associated virus (LAV) or AIDS-related virus (ARV) or human immunodeficiency virus (HIV). Up until now, two distinct families have been identified, i.e. HIV-1 and HIV-2. Hereinafter, HIV will be used to generically denote these viruses.

One of the critical pathways in a retroviral life cycle is the processing of polyprotein precursors by retroviral protease. For instance, during the replication cycle of the HIV virus, gag and gag-pol gene transcription products are translated as proteins, which are subsequently processed by a virally encoded protease to yield viral enzymes and structural proteins of the virus core. Most commonly, the gag precursor proteins are processed into the core proteins and the pol precursor proteins are processed into the viral enzymes, e.g., reverse transcriptase and retroviral protease. Correct processing of the precursor proteins by the retroviral protease is necessary for the assembly of infectious virions, thus making the retroviral protease an attractive target for antiviral therapy. In particular for HIV treatment, the HIV protease is an attractive target.

Several protease inhibitors are on the market or are being developed. Hydroxyethylamino sulfonamide HIV protease inhibitors, for example 4-aminobenzene hydroxyethylamino sulfonamides, have been described to have favourable pharmacological and pharmacokinetic properties against wild-type and mutant HIV virus. Amprenavir is a commercially available exponent of this 4-aminobenzene hydroxyethylamino sulfonamide class of protease inhibitors.

One such protease inhibitor which has been approved in the USA for human clinical use for the treatment of retroviral infections and having the above structural moiety is the compound having the USAN approved name darunavir with the chemical name [(1S,2R)-3-[[(4-aminophenyl)sulfonyl](2-methylpropyl)amino]-2-hydroxy-1-(phenylmethyl)propyl]-carbamic acid (3R,3aS,6aR)hexahydrofuro[2,3-b]furan-3-yl ester, in the form of the ethanolate derivate, and the structure of formula (A):

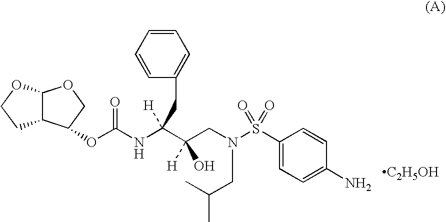

Unless otherwise specified, the term “darunavir” will be used herein to indicate the parent compound or a derivative thereof such as a hydrate or a solvate especially an alcoholate for example the ethanolate.

Darunavir in the form of its ethanolate is generally administered to patients in a tablet formulation and a tablet formulation containing 300 mg of the darunavir parent compound has been developed and marketed by the applicants for treatment-experienced HIV patients (i.e. who have previously received anti-HIV chemotherapy) in the recommended dosage of 1200 mg per day (two 300 mg tablets twice daily). The 300 mg tablet comprises 52.02% by weight (w/w) of darunavir (as the ethanolate), 45.74% w/w of a filler (a co-processed spray-dried mixture of 98% w/w microcrystalline cellulose and 2% w/w colloidal silicon dioxide, available commercially as PROSOLV SMCC HD90), 2% w/w of a disintegrant (crospovidone) and 0.24% w/w of a lubricant (magnesium stearate). The above tablet core is film-coated with an Opadry film-coat. The tablet is manufactured by dry blending of the above core ingredients followed by compression and then film-coating.

The applicants are also developing and investigating in the clinic tablets of darunavir ethanolate for administration to so-called naïve patients who have not hitherto received any anti-HIV therapy. These tablets contain 400 mg of darunavir parent compound for administration twice a day to provide a total daily dosage of 800 mg of the compound. Alternative tablets being developed include those containing 800 mg of darunavir parent compound for administration once a day. Pediatric tablet formulations containing 75 mg and 150 mg of darunavir parent compound are also being investigated for administration to children.

The active ingredient darunavir ethanolate used in the marketed 300 mg tablet formulation has been manufactured by a process which includes a drying process in which the particulate darunavir is dried on trays, i.e. a static process. However, in order to provide a higher manufacturing throughput and improved safety for the operators, it is desirable to replace the static drying with a tumble drying operation involving drying in a revolving container. Such a change in the drying operation however has a significant effect on the physical characteristics of the particles of the darunavir. In this case the tumble drying of the particles results in a reduction in the dv10 value (dv10 means that 10% (volume %) of the particles has a diameter smaller than the specified value) from approximately 80 microns to approximately 35 microns. The smaller particle size in turn affects the flow properties of the drug product dry blend, notably an increased tendency to caking or agglomeration of the particles and thus reduced flow characteristics. Experimental runs on a pilot scale showed a reduced drug product blend flow capacity, tablet weight variation and tablet sticking when using tumble dried darunavir in the currently marketed formulation. It will be appreciated that good blend flow and good tablet properties such as weight uniformity and appropriate visual appearance, without any defects, are essential in a large-scale industrial manufacturing process to maximise throughput and ensure consistent and efficient processing of the material.

As will be apparent from the above discussion the currently marketed darunavir tablet formulation has been found to be unsuitable for manufacture on a larger scale when the drying of the active ingredient needs to be carried out by the faster tumble drying. There is therefore a need for a tablet formulation which can be manufactured using darunavir having a smaller particle size and a reduced flowability resulting from a tumble drying process. The problems of optimising the flowability of powders are described for example in Pharmaceutical Technology, Feb. 2, 2006 (Maribel Ros) and LabPlus International February/March 2004, Volume 18, No. 1, 8-10.

When developing tablet formulations for manufacture on a commercial scale it is therefore necessary to ensure that the manufacturing process can be carried out economically and efficiently in high throughput and with excellent uniformity of product.

### DESCRIPTION OF THE INVENTION

It is an object of the present invention to provide an improved tablet formulation containing darunavir, which tablet formulation can be manufactured on an industrial scale.

It is an object of the present invention to provide a tablet formulation containing darunavir, which can be manufactured on an industrial scale with improved efficiency.

It is an object of the present invention to provide a tablet formulation containing darunavir, which can be manufactured on an industrial scale with improved safety for the process operators.

It is an object of the present invention to provide a tablet formulation containing darunavir, which can be manufactured on an industrial scale with improved throughput.

It is an object of the present invention to provide a tablet formulation containing darunavir generated from a tumble drying process.

It is an object of the present invention to provide a tablet formulation having overall improved flow properties.

It is an object of the present invention to provide a process for the manufacture of an improved darunavir tablet formulation.

We have found that significant improvements in the flow characteristics of the above formulations can be achieved by the use of additional colloidal silicon dioxide as a separate component and an increased amount of lubricant. These formulation changes provide a tablet manufacturing process that is more robust towards potential variations in the flow properties specific to the active ingredient, in that it allows for the use of an active ingredient with a smaller particle size and reduced flow properties.

According to the present invention therefore we provide a tablet formulation comprising a tablet core containing 0.1 to 1.5% by weight (w/w) of colloidal silicon dioxide and 0.4 to 0.9% by weight (w/w) of a lubricant, the balance of the core comprising darunavir, a disintegrant and a filler comprising a spray-dried mixture of microcrystalline cellulose and colloidal silicon dioxide, the core being optionally coated with a film coating.

We have found that the use of additional colloidal silicon dioxide, i.e. discrete from that contained in the PROSOLV (a spray-dried mixture of 98% w/w microcrystalline cellulose and 2% w/w colloidal silicon dioxide) material used in the above 300 mg formulation, provides benefits not achieved by the use of colloidal silicon dioxide contained solely within the spray-dried mixture. From experiments conducted by the applicants it appears that the colloidal silicon dioxide within the spray-dried mixture provides very good compressibility, but does not provide sufficient anti-caking and flow-enhancing properties in the proposed tablet formulations. Moreover, further experiments by the applicants have also established that the use of microcrystalline cellulose and colloidal silicon dioxide as separate discrete ingredients also does not provide a tablet formulation having optimum flow properties. Thus, the applicants have found that it is necessary to include in the tablet formulation not only the spray-dried microcrystalline cellulose/colloidal silicon dioxide mixture but also additional colloidal silicon dioxide as a separate component in order to achieve the benefits typically associated with colloidal silicon dioxide, i.e. enhanced flow and reduced caking tendency of the drug product blend.

The additional colloidal silicon dioxide is generally present in the tablet formulations according to the invention in an amount of 0.3 to 1.1% w/w, preferably 0.5 to 1.1% w/w, for example about 0.9% w/w, particularly about 0.91% w/w. The colloidal silicon dioxide which is advantageously employed in the tablet formulations according to the invention is that which is commercially available as Cab-O-Sil, particularly the M5P grade.

In addition to the presence of colloidal silicon dioxide as a separate ingredient, the tablet formulation according to the invention contains an increased amount of lubricant over that in the marketed 300 mg tablet formulation, providing a formulation which avoids manufacturing problems such as tablet sticking when the drug product blend is compressed into tablets.

The lubricant is preferably magnesium stearate and is generally present in an amount of 0.5 to 0.8% w/w, particularly about 0.7% w/w, especially about 0.74% w/w.

The tablet formulation according to the invention further contains a filler comprising a spray-dried mixture of microcrystalline cellulose and colloidal silicon dioxide. This filler is advantageously one comprising a mixture of about 98% w/w of microcrystalline cellulose and about 2% w/w of colloidal silicon dioxide, for example the mixture available commercially as PROSOLV, especially the HD90 product, as used in the 300 mg tablet formulation referred to above. The mixture is generally present in the tablet formulation according to the invention in an amount of 40 to 50% w/w, preferably 43 to 46% w/w and especially about 44% w/w, particularly about 44.33% w/w.

Darunavir is generally present in the tablet formulations according to the invention in an amount of 50 to 55% w/w preferably 51 to 53% w/w, especially about 52% w/w and particularly 52.02% w/w.

The darunavir is generally employed in the tablet formulations according to the invention in the form of a crystalline derivative of the darunavir parent compound such as a hydrate or solvate for example an alcoholate, the ethanolate being especially preferred.

As discussed previously the tablet formulations according to the invention enable one to prepare tablets using darunavir with a smaller particle size than was possible with the previous 300 mg formulation. Thus the darunavir in the tablets according to the invention generally contains darunavir with a dv10 value in the range of 12 to 102 microns and a dv50 value in the range of 47 to 249 microns.

The tablet formulation also contains a disintegrant to aid disintegration and dissolution of the formulation upon administration to the patients. The preferred disintegrant is crospovidone, namely a synthetic homopolymer of cross-linked N-vinyl-2-pyrrolidone available commercially as Polyplasdone XL-10 and is preferably present in an amount of 1 to 3% w/w, especially about 2% w/w. Other disintegrants which may be used include croscarmellose sodium (sodium salt of cross-linked carboxymethylcellulose), available commercially as Acdisol.

The new tablet formulation can be used in a dose-proportional way to prepare tablets containing different amounts of the active ingredient darunavir to facilitate administration of the tablets depending upon the dosage of darunavir prescribed. Thus tablets can be prepared containing for example 75 and 150 mg (for pediatric use), 400 and 800 mg (for patients who have not previously received anti-HIV treatment) and 600 mg of darunavir per tablet for patients who have previously received anti-HIV treatment; the amounts of darunavir in the tablet cores are based on the weight of parent darunavir compound.

For the purpose of illustration, specific examples of the 600 mg, 75 mg and 400 mg tablet core formulations are shown below:

| Ingredients | 600 mg tablet | | 75 mg tablet | | 400 mg tablet | |
|---|---|---|---|---|---|---|
| Darunavir (as ethanolate) | 650.46 | | 81.31 | | 433.64 | |
| Prosolv | 554.30 | | 69.28 | | 369.53 | |
| Colloidal silicon dioxide | 11.38 | | 1.42 | | 7.59 | |
| Crospovidone | 25.01 | | 3.13 | | 16.67 | |
| Magnesium stearate | 9.25 | | 1.16 | | 6.17 | |
| TOTAL | 1250.4 | mg | 156.3 | mg | 833.6 | mg |

For taste-masking and cosmetic reasons the tablet cores according to the invention are generally provided with a film coating for example an Opadry film-coating, which is generally used in an amount of about 4% w/w based on the tablet core. Different colouring agents may be used in the film coating in order to differentiate between tablet strengths.

The above tablet formulations can be used to make tablet cores in conventional manner for example by initially dry blending the darunavir, the spray-dried microcrystalline cellulose/colloidal silicon dioxide mixture, the additional colloidal silicon dioxide and the disintegrant, the ingredients preferably having been sieved, and then adding the lubricant, which has preferably also been sieved, to the dry-blended mixture for final dry-blending of the total tablet core blend which is then compressed into tablets having the desired size and weight.

After preparation of the tablet core as described above the core can be film-coated in conventional manner for example by film-coating with a film-coating agent such as Opadry which can be applied to the core in a coating suspension for example in purified water, followed by drying of the coated cores.

As indicated above the tablet formulations according to the invention can be used in the treatment of HIV infections.

According to a further feature of the invention we provide the tablet formulations according to the invention for use in medicine for example for the treatment of HIV infections.

According to a further feature of the invention we provide a method for the treatment of an HIV infection in a subject which comprises administering to the subject an effective amount of a tablet formulation according to the invention.

In accordance with the invention the tablet formulations can be employed for the treatment of HIV infections in various dosages depending on the age and clinical status of the patient. Thus for example, for administration to patients who have not hitherto received any anti-HIV therapy tablets containing 400 mg of darunavir (parent compound) can be administered twice a day to provide a total daily dosage of 800 mg. For administration to patients who have previously received anti-HIV therapy tablets containing 600 mg of darunavir (parent compound) can be administered twice a day to provide a total daily dosage of 1200 mg.

Darunavir can be administered to HIV patients in combination with other anti-HIV compounds such as, for instance nucleoside reverse transcriptase inhibitors (NRTIs), non-nucleoside reverse transcriptase inhibitors (NNRTIs) or other protease inhibitors.

Some antiretrovirals and, in particular, some HIV protease inhibitors such as darunavir are metabolized by cytochrome P₄₅₀, leading to sub-optimal pharmacokinetic profiles, causing an undesired need for more frequent and higher doses. It is therefore desirable for darunavir to be administered in combination with an inhibitor of cytochrome P₄₅₀. Examples of inhibitors of cytochrome P₄₅₀ which are also HIV protease inhibitors include for example ritonavir, indinavir, nelfinavir, saquinavir, amprenavir, lopinavir, lasinavir, palinavir, telinavir, tipranavir, mozenavir, atazanavir and pharmaceutically acceptable salts and esters thereof. More particularly, the cytochrome P₄₅₀ inhibitor is selected from the group comprising ritonavir, amprenavir, nelfinavir or a pharmaceutically acceptable salt or ester thereof, ritonavir being especially preferred. Combinations of protease inhibitors such as darunavir and cytochrome P₄₅₀ inhibitor such as ritonavir are described and claimed in patent specification WO03/049746, the contents of which are incorporated herein by reference.

The cytochrome P₄₅₀ inhibitor such as ritonavir is generally administered in combination with darunavir in a dosage of 100 mg bid.

The individual components of the combination of the present invention, namely darunavir and the cytochrome P₄₅₀ inhibitor can be administered separately at different times during the course of therapy or concurrently in divided or single combination forms.

The following examples are meant to illustrate the present invention. The examples are presented to exemplify the invention and are not to be considered as limiting the scope of the invention.

### Examples

A) 600 mg Tablet Formulation

For the manufacture of a typical 115.5 kg batch of the 600 mg tablets in accordance with the invention following steps are carried out:

1. Darunavir as the ethanolate: 60.08 kg, Prosolv HD90: 51.20 kg; colloidal silicon dioxide (Cab-O-Sil): 1.05 kg; crospovidone (POLYPLASDONE XL-10): 2.31 kg; and magnesium stearate: 0.86 kg, are weighed out to provide a batch with a total weight of 115.5 kg.
2. The weighed out materials from Step 1, except for the magnesium stearate, are loaded in a bin.
3. The darunavir, colloidal silicon dioxide, crospovidone and Prosolv in the bin are delumped by passage through a 11 to 16 mesh screen in a suitable sieving apparatus and the delumped mixture is collected in a second bin.
4. The bin is transported to a bin blender, where the dry mixture is blended for 10 minutes at 6 rpm (rotations per minute).
5. Magnesium stearate is sieved through a 18 to 20 mesh screen in a suitable sieving apparatus and then added on top of the blended material in the bin from Step 4.
6. After addition of the magnesium stearate a final blending of the mixture is performed for 5 minutes at 6 rpm.
7. The resulting blend is compressed into tablets in a conventional tablet press to provide a batch of 92,370 tablet cores with a core weight of 1250.4 mg.
8. The resulting tablet cores are film-coated in a film coating machine with a total of 28.88 kg of a coating suspension comprising 5.78 kg of Opadry II Orange and 23.10 kg of Purified Water to provide coated tablets with a total tablet weight of 1300.4 mg and containing 650.46 mg of darunavir ethanolate, 600 mg as the parent compound.

B) 400 mg Tablet Formulation

The procedure described in Steps 1 to 6 of Part A) is repeated and the resultant common blend is then compressed in a conventional tablet press to provide a batch of 138,555 tablet cores with a core weight of 833.6 mg. The tablet cores are then film coated in an analogous manner to the procedure described in Step 8 in Part A) to provide film coated tablets with a total tablet weight of 866.9 mg and containing 433.64 mg of darunavir ethanolate, 400 mg as the parent compound.

C) 75 mg Tablet Formulation

The procedure described in Steps 1 to 6 of Part A) is repeated and the resultant common blend is then compressed in a conventional tablet press to provide a batch of 738,963 tablets with a core weight of 156.3 mg. The tablet cores are then film coated in accordance with the procedure described in Step 8 in Part A) to provide film coated tablets with a total tablet weight of 162.6 mg and containing 81.31 mg of darunavir ethanolate, 75 mg as the parent compound.

The above tablet formulations were evaluated for ease of manufacture on an industrial scale and were found to have excellent properties both in terms of blend flow, namely an angle of repose of approximately 45° , mass flow characteristics in a Gurabo bin (with a bin angle of 30° from vertical) and a low Carr index of about 16, and also tablet characteristics, namely a tablet weight variation below 1% for relative standard deviation, a smooth, shining tablet surface and low friability of about 0.1%.

1. A tablet formulation comprising a core containing 0.1 to 1.5% by weight (w/w) of colloidal silicon dioxide and 0.4 to 0.9% by weight (w/w) of a lubricant, the balance of the core comprising darunavir, a disintegrant and a filler comprising a spray-dried mixture of microcrystalline cellulose and colloidal silicon dioxide, the core being optionally coated with a film coating.

2. A tablet formulation as claimed in claim 1 in which the core comprises 50 to 55% w/w of darunavir.

3. (canceled)

4. A tablet formulation as claimed in claim 1 in which the core comprises about 52% w/w of darunavir.

5. (canceled)

6. A tablet formulation as claimed in claim 1 in which the darunavir is present in the form of its ethanolate.

7. (canceled)

8. A tablet formulation as claimed in claim 1 in which the core comprises 0.5 to 1.1% w/w of colloidal silicon dioxide in addition to that contained in the said filler.

9. A tablet formulation as claimed in claim 8 in which the core comprises about 0.9% w/w of colloidal silicon dioxide in addition to that contained in the said filler.

10. (canceled)

11. A tablet formulation as claimed in claim 1 in which the core comprises 0.5 to 0.8% w/w of the lubricant.

12.-13. (canceled)

14. A tablet formulation as claimed in claim 1 in which the lubricant is magnesium stearate.

15. A tablet formulation as claimed in claim 1 in which the filler comprises a mixture of about 98% w/w of microcrystalline cellulose and about 2% w/w colloidal silicon dioxide.

16. A tablet formulation as claimed in claim 1 in which the core comprises 40 to 50% w/w of the filler.

17.-18. (canceled)

19. A tablet formulation as claimed in claim 1 in which the core comprises 1 to 3% w/w of the disintegrant.

20. (canceled)

21. A tablet formulation as claimed in claim 1 in which the disintegrant is crospovidone.

22. A tablet formulation as claimed in claim 1 in which the darunavir in the core has a dv10 particle size value in the range of 12 to 102 microns.

23. A tablet formulation as claimed in claim 22 in which the darunavir in the core has a dv10 particle size value in the range of 21 to 77 microns

24. A tablet formulation as claimed in claim 23 in which the darunavir in the core has a dv10 particle size value in the range of 21 to 59 microns.

25. A process for preparing a tablet formulation as claimed in claim 1 which comprises dry blending a mixture of the darunavir, the spray-dried microcrystalline/colloidal silicon dioxide mixture, the additional colloidal silicon dioxide and the disintegrant, adding the lubricant to the dry-blended mixture for final dry-blending of the total core composition which is then compressed to provide tablet cores, the said cores then being optionally film-coated.

26.-27. (canceled)

28. A method for the treatment of an HIV infection in a subject which comprises administering to the subject an effective amount of a tablet formulation as claimed in claim 1.
